(19) United States
(12) Patent Application Publication
(10) Pub. No.: US 2026/0137299 A1
(43) Pub. Date: May 21, 2026

(54) SYSTEMS AND METHODS FOR AUTHENTICATION USING SOUND-BASED VOCALIZATION ANALYSIS

(71) Applicant: Covid Cough, Inc., Greenwood Village, CO (US)

(72) Inventors: Nolan Donaldson, Englewood, CO (US); Mark Fogarty, Seneca, SC (US); Kristan S. Hopkins, North Andover, MA (US); Simon Kotchou, Phoenix, AZ (US); Robert Scordia, Ridgewood, NY (US); Adam Stogsdill, Cypress, TX (US); Seraphim Therianos, Foxfield, CO (US)

(73) Assignee: Covid Cough, Inc., Greenwood Village, CO (US)

(21) Appl. No.: 19/110,288

(22) PCT Filed: Sep. 15, 2023
(86) PCT No.: PCT/US2023/074299

§ 371 Date: Mar. 10, 2025

Related U.S. Application Data
(60) Provisional application No. 63/375,818, filed on Sep. 15, 2022.

(51) Int. Cl. A61B 5/08; A61B 5/00; G10L 25/66

(52) U.S. Cl. CPC A61B 5/0823; A61B 5/7264; G10L 25/66

(57) ABSTRACT

Systems and methods use a processor to obtain a signal data signatures (SDS) of a forced cough vocalizations (FCV). The processor controls a display present an instruction to a user to produce an FCV. The processor controls a recording device to record audio during the FCV so as to receive an audio signal that captures the FCV. The processor pre-processes the audio signal and outputs FCV signal data representative of the FCV. The processor uses a cough signature model to ingest the FCV signal data and output an SDS representative of isolated FCV-related signal data. The processor validates one or more SDSs by classifying and testing the SDS against SDS criteria (baseline) that establish a minimum quality7 of the SDS. Based on the SDS failing to achieve the SDS criteria, the processor deletes the SDS and controls the display to present a new instruction to the user for a new FCV.

### CLAIM TO PRIORITY

This application claims priority to U.S. Provisional Application Number 63/375,818 filed on 15 Sep. 2022 and entitled “SYSTEMS AND METHODS FOR AUTHENTICATION USING SOUND-BASED VOCALIZATION ANALYSIS,” and is herein incorporated by reference in its entirety.

### FIELD OF TECHNOLOGY

The present invention relates generally to Artificial Intelligence and specifically to audio segmentation and feature extraction for Signal Data Signature (SDS) classification based on forced cough vocalizations.

### BACKGROUND OF TECHNOLOGY

The application of biometric and physiologic source signal data signature detection as a respiratory health diagnostic or screening tool is particularly attractive as it represents a non-intrusive, real-time diagnostic that can be essential to respiratory health and wellbeing tracking.

Typically, systems are limited to complex systems that have been created to identify, monitor, and record spontaneous cough. Auscultation of a spontaneous cough is difficult in that it must be timed, coughs are not on demand, and often are accompanied by background noise.

### SUMMARY OF DESCRIBED SUBJECT MATTER

In some aspects, the techniques described herein relate to a method including: controlling, by at least one processor, a display of a computing device to render a forced cough vocalization graphical user interface (GUI) including at least one user instruction instructing the user to auscultate and produce a forced cough vocalization; controlling, by the at least one processor, a recording device associated with the computing device to record audio; receiving, by the at least one processor from the recording device, at least one audio signal that captures the forced cough vocalization produced by the user; utilizing, by the at least one processor, at least one pre-processing algorithm to pre-process the at least one audio signal and output forced cough vocalization signal data representative of the forced cough vocalization; utilizing, by the at least one processor, at least one cough signature model to ingest the forced cough vocalization signal data and output a signal data signature representative of isolated forced cough vocalization-related signal data isolated from non-forced cough vocalization-related signal data; wherein the at least one cough signature model to ingests the forced cough vocalization signal data and outputs the signal data signature based at least in part on at least one machine learning layer including a plurality of machine learning parameters; wherein the plurality of machine learning parameters is iteratively refined based on error between: previous predictions based on historical signal data signatures and known signal data signatures paired with the historical signal data signatures; determining, by the at least one processor, a signal data signature invalidation based at least in part on testing of the signal data signature against at least one signal data signature criteria resulting in the signal data signature failing to achieve the at least one signal data signature criteria; deleting, by the at least one processor, based on the signal data signature invalidation, the signal data signature; and controlling, by the at least one processor, the display of the computing device to render the forced cough vocalization GUI including at least one subsequent user instruction instructing the user to auscultate and produce a new forced cough vocalization in response to the signal data signature invalidation.

In some aspects, the techniques described herein relate to a method, further including: controlling, by the at least one processor, the recording device associated with the computing device to record new audio; receiving, by the at least one processor from the recording device, at least one new audio signal that captures the new forced cough vocalization produced by the user; utilizing, by the at least one processor, the at least one pre-processing algorithm to pre-process the at least one new audio signal and output new forced cough vocalization signal data representative of the new forced cough vocalization; utilizing, by the at least one processor, the at least one cough signature model to ingest the new forced cough vocalization signal data and output a new signal data signature representative of new isolated forced cough vocalization-related signal data isolated from new non-forced cough vocalization-related signal data; determining, by the at least one processor, a signal data signature validation based at least in part on testing of the new signal data signature against the at least one signal data signature criteria resulting in the signal data signature achieving the at least one signal data signature criteria; and uploading, by the at least one processor, the new signal data signature to a remote cough analysis service configured to utilize at least one machine learning model to detect at least one anomaly in the new signal data signature.

In some aspects, the techniques described herein relate to a method, wherein the remote cough analysis service further configured to store the new signal data signature in a signal data signature library associated with the user so as to produce a set of baseline signal data signatures.

In some aspects, the techniques described herein relate to a method, wherein the at least one signal data signature criteria includes at least one of: a maximum signal-to-noise ratio, a maximum noise floor, a minimum audio quality associated with the signal data signature, a hardware failure, a network error, a cough sound or forced cough(s) and a phasing criteria.

In some aspects, the techniques described herein relate to a method, wherein the hardware failure includes at least one of: a failure in operation of the recording device, and a failure in operation of a storage device associated with storing the at least one audio signal.

In some aspects, the techniques described herein relate to a method, wherein the at least one processor is local to the computing device.

In some aspects, the techniques described herein relate to a method, further including: utilizing, by the at least one processor, the at least one cough signature model to ingest the forced cough vocalization signal data and output a plurality of signal data signatures representative of isolated forced cough vocalization-related signal data isolated for a plurality of forced cough vocalizations in the at least one audio signal.

In some aspects, the techniques described herein relate to a system including: at least one processor configured to execute software instructions that, upon execution, cause the at least one processor to perform steps to: control a display of a computing device to render a forced cough vocalization graphical user interface (GUI) including at least one user instruction instructing the user to auscultate and produce a forced cough vocalization; control a recording device associated with the computing device to record audio; receive, from the recording device, at least one audio signal that captures the forced cough vocalization produced by the user; utilize at least one pre-processing algorithm to pre-process the at least one audio signal and output forced cough vocalization signal data representative of the forced cough vocalization; utilize at least one cough signature model to ingest the forced cough vocalization signal data and output a signal data signature representative of isolated forced cough vocalization-related signal data isolated from non-forced cough vocalization-related signal data; wherein the at least one cough signature model to ingests the forced cough vocalization signal data and outputs the signal data signature based at least in part on at least one machine learning layer including a plurality of machine learning parameters; wherein the plurality of machine learning parameters is iteratively refined based on error between: previous predictions based on historical signal data signatures and known signal data signatures paired with the historical signal data signatures; determine a signal data signature invalidation based at least in part on testing of the signal data signature against at least one signal data signature criteria resulting in the signal data signature failing to achieve the at least one signal data signature criteria; delete based on the signal data signature invalidation, the signal data signature; and control the display of the computing device to render the forced cough vocalization GUI including at least one subsequent user instruction instructing the user to auscultate and produce a new forced cough vocalization in response to the signal data signature invalidation.

In some aspects, the techniques described herein relate to a system including user instruction that may consist of audible and/or visual cues to a user on the recording device in order to commence and execute one or more forced cough vocalizations. In some aspects, the disclosed system can involve, provide and/or request, but is not limited to, a recommended breathing duration and timing prior to one or more FCVs, and if multiple FCVs, the timing of consecutive FCVs to be captured within the a single SDS.

In some aspects, the techniques described herein relate to a system, wherein, upon execution of the software instructions, the at least one processor is further configured to: control the recording device associated with the computing device to record new audio; receive, from the recording device, at least one new audio signal that captures the new forced cough vocalization produced by the user; utilize the at least one pre-processing algorithm to pre-process the at least one new audio signal and output new forced cough vocalization signal data representative of the new forced cough vocalization; utilize the at least one cough signature model to ingest the new forced cough vocalization signal data and output a new signal data signature representative of new isolated forced cough vocalization-related signal data isolated from new non-forced cough vocalization-related signal data; determine a signal data signature validation based at least in part on testing of the new signal data signature against the at least one signal data signature criteria resulting in the signal data signature achieving the at least one signal data signature criteria; and upload the new signal data signature to a remote cough analysis service configured to utilize at least one machine learning model to detect at least one anomaly in the new signal data signature.

In some aspects, the techniques described herein relate to a system, wherein the remote cough analysis service further configured to store the new signal data signature in a signal data signature library associated with the user so as to produce a set of baseline signal data signatures.

In some aspects, the techniques described herein relate to a system, wherein the at least one signal data signature criteria includes at least one of: a maximum signal-to-noise ratio, a maximum noise floor, a minimum audio quality associated with the signal data signature, a hardware failure, a network error, a cough sound or forced cough(s) and a phasing criteria.

In some aspects, the techniques described herein relate to a system, wherein the hardware failure includes at least one of: a failure in operation of the recording device, and a failure in operation of a storage device associated with storing the at least one audio signal.

In some aspects, the techniques described herein relate to a system, wherein the at least one processor is local to the computing device.

In some aspects, the techniques described herein relate to a system, wherein, upon execution of the software instructions, the at least one processor is further configured to: utilize the at least one cough signature model to ingest the forced cough vocalization signal data and output a plurality of signal data signatures representative of isolated forced cough vocalization-related signal data isolated for a plurality of forced cough vocalizations in the at least one audio signal.

In some aspects, the techniques described herein relate to a non-transitory computer readable medium having software instructions stored thereon, the software instructions configured to cause at least one processor to perform steps including: control a display of a computing device to render a forced cough vocalization graphical user interface (GUI) including at least one user instruction instructing the user to produce a forced cough vocalization for the purpose of auscultation by a recording device and system; control a recording device associated with the computing device to record audio; receive, from the recording device, at least one audio signal that captures the forced cough vocalization produced by the user; utilize at least one pre-processing algorithm to pre-process the at least one audio signal and output forced cough vocalization signal data representative of the forced cough vocalization; utilize at least one cough signature model to ingest the forced cough vocalization signal data and output a signal data signature representative of isolated forced cough vocalization-related signal data isolated from non-forced cough vocalization-related signal data; wherein the at least one cough signature model to ingests the forced cough vocalization signal data and outputs the signal data signature based at least in part on at least one machine learning layer including a plurality of machine learning parameters; wherein the plurality of machine learning parameters is iteratively refined based on error between: previous predictions based on historical signal data signatures and known signal data signatures paired with the historical signal data signatures; determine a signal data signature invalidation based at least in part on testing of the signal data signature against at least one signal data signature criteria resulting in the signal data signature failing to achieve the at least one signal data signature criteria; delete based on the signal data signature invalidation, the signal data signature; and control the display of the computing device to render the forced cough vocalization GUI including at least one subsequent user instruction instructing the user to auscultate and produce a new forced cough vocalization in response to the signal data signature invalidation.

In some aspects, the techniques described herein relate to a non-transitory computer readable medium, wherein the software instructions are further configured to cause the at least one processor to perform steps including: control the recording device associated with the computing device to record new audio; receive, from the recording device, at least one new audio signal that captures the new forced cough vocalization produced by the user; utilize the at least one pre-processing algorithm to pre-process the at least one new audio signal and output new forced cough vocalization signal data representative of the new forced cough vocalization; utilize the at least one cough signature model to ingest the new forced cough vocalization signal data and output a new signal data signature representative of new isolated forced cough vocalization-related signal data isolated from new non-forced cough vocalization-related signal data; determine a signal data signature validation based at least in part on testing of the new signal data signature against the at least one signal data signature criteria resulting in the signal data signature achieving the at least one signal data signature criteria; and upload the new signal data signature to a remote cough analysis service configured to utilize at least one machine learning model to detect at least one anomaly in the new signal data signature.

In some aspects, the techniques described herein relate to a non-transitory computer readable medium, wherein the remote cough analysis service further configured to store the new signal data signature in a signal data signature library associated with the user so as to produce a set of baseline signal data signatures.

In some aspects, the techniques described herein relate to a non-transitory computer readable medium, wherein the at least one signal data signature criteria includes at least one of: a maximum signal-to-noise ratio, a maximum noise floor, a minimum audio quality associated with the signal data signature, a hardware failure, a network error, a cough sound or forced cough(s) and a phasing criteria.

In some aspects, the techniques described herein relate to a non-transitory computer readable medium, wherein the hardware failure includes at least one of: a failure in operation of the recording device, and a failure in operation of a storage device associated with storing the at least one audio signal.

In some aspects, the techniques described herein relate to a non-transitory computer readable medium, wherein the software instructions are further configured to cause the at least one processor to perform steps including: utilize the at least one cough signature model to ingest the forced cough vocalization signal data and output a plurality of signal data signatures representative of isolated forced cough vocalization-related signal data isolated for a plurality of forced cough vocalizations in the at least one audio signal.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1A is a block diagram of an exemplary computer-based system for sound signal data signature analysis for vocalization-based authentication utilizing machine learning in accordance with one or more embodiments of the present disclosure.

FIG. 1B illustrates phases of a forced cough vocalization in an audio signal in accordance with one or more embodiments of the present disclosure.

FIG. 2 depicts an illustrative computer-based system for sound signal data signature analysis including a sound signal data signature analysis service that receives sound signal data signatures from a user computing device according to embodiments of the present disclosure.

FIG. 3 depicts an illustrative computer-based system for sound signal data signature analysis including a user computing device configured to record sound signal data signatures of a user according to embodiments of the present disclosure.

FIG. 4 depicts an illustrative AI sound signal data signature analysis model engine of the sound signal data signature analysis service for sound signal data signature analysis according to embodiments of the present disclosure.

FIG. 5 depicts an illustrative system for the SDS recording engine 120 including one or more components configured for SDS validation to validate the SDS in accordance with one or more embodiments of the present disclosure.

FIG. 6 depicts a block diagram of an exemplary computer-based system and platform 700 in accordance with one or more embodiments of the present disclosure.

FIG. 7 depicts a block diagram of another exemplary computer-based system and platform 800 in accordance with one or more embodiments of the present disclosure.

FIG. 8 illustrates schematics of an exemplary implementation of the cloud computing/architecture(s) in which the computer-based systems of the present disclosure may be specifically configured to operate.

FIG. 9 illustrates schematics of another exemplary implementation of the cloud computing/architecture(s) in which the computer-based systems of the present disclosure may be specifically configured to operate.

### DETAILED DESCRIPTION

Each human being has a very distinct forced non-speech vocalization (aka fake cough) that technology can measure and identify as your baseline sound signal data signature. By analyzing a sound signal data signature against a pre-recorded baseline sound signal data signature, some embodiments may provide an early warning if the sound signal data signature no longer matches the baseline sound signal data signature.

Accordingly, in some embodiments, identifying potential respiratory anomalies for early warning using sound signal data signatures may include a sound signal data signature recording step, an artificial intelligence (AI) driven sound signal data signature analysis to determine the sound signal data signature, and an AI driven sound signal data signature identification to identify whether the sound signal data signature matches a baseline sound signal data signature.

In particular, the present disclosure provides systems and methods for signal data signature classification, including the capture of signal data signature segments in order to allow for storage, classification and evaluation of the source signal data signature and source signal data signature segments.

In some embodiments, the systems and methods of the present disclosure utilize a forced cough vocalization and auscultated sounds that each independently or collectively are used for developing, calibrating, adjudicating, training, validating, verifying, testing, and developing of artificial intelligence (AI)/machine learning (ML) based software, such as AI/ML based software for medical device (SaMD) systems and other signal data signature based AI/ML systems.

In some embodiments, users may utilize a software application, such as, e.g., a native application, a web application, a browser page, or any other suitable application (“app”) or any combination thereof. The users may download or otherwise access the app and submit a recording of a sound signal data signature to a computing device, such as, e.g., a personal computing device or computing system (e.g., laptop computer, desktop computer, etc.), a mobile computing device (e.g., a smartphone, tablet, wearable, etc.), a cloud service, or other computing device or computing system to establish a baseline. In some embodiments, to establish the baseline sound signal data signature, the user may submit sound signal data signatures multiples times, such as, e.g., two times, three times, five times, or other suitable number of times. Thereafter, a baseline sound signal data signature may be determined from the multiple sound signal data signatures such that a new sound signal data signature can be tested against the baseline sound signature, e.g., for biometric authentication purposes, diagnostic purposes, health tracking, or any other suitable downstream processing based on the unique respiratory characteristics elicited by a user's forced cough vocalization. In some embodiments, the software application used and executed according to the disclosed systems and methods can provide visual and/or audio cues via the user interface to illicit one or more FCVs at a particular time that the recording device can utilize to optimally capture the audio.

In some embodiments, the software application used and executed according to the disclosed systems and methods can provide visual and/or audio cues in the user interface to illicit breathing and/or breath timing prior to one or more FCVs at a particular time that the recording device can optimally utilize to capture the audio.

In some embodiments, the software application used and executed according to the disclosed systems and methods can provide visual and/or audio cues in the user interface to provide timing of multiple FCVs at a particular time that the recording device can optimally utilize to capture the audio.

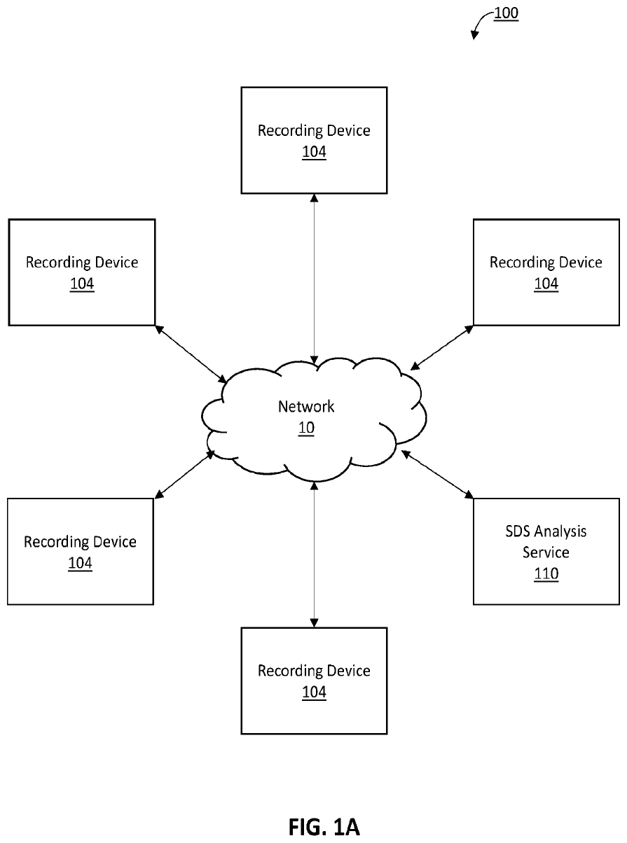

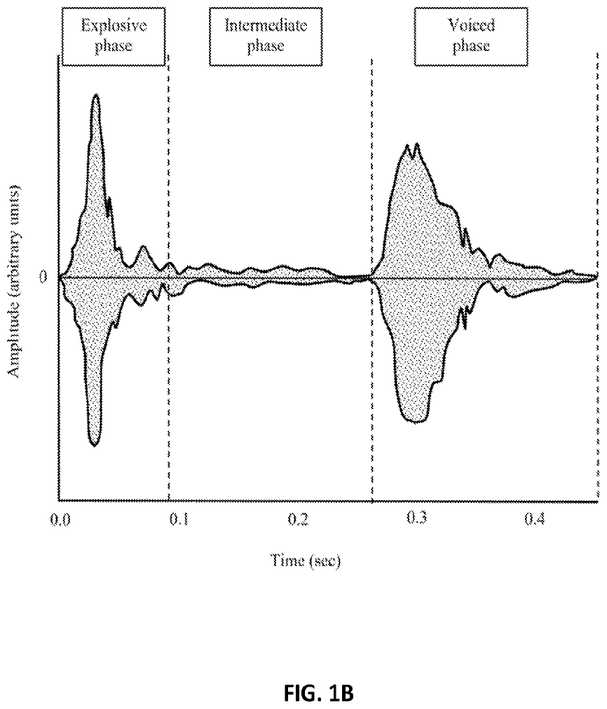

FIG. 1A is a block diagram of an exemplary computer-based system for sound signal data signature analysis in accordance with one or more embodiments of the present disclosure.

During the initial compressive phase of a FCV, the glottis closes and the abdominal muscles contract, forcibly increasing air pressure in the lungs and chest cavity against the closed glottis. Then the vocal cords open and air rushes out very quickly, expelling mucus and particles in the airways. All phases of the FCV are the symphonic composition of the sounds originating and resonating throughout the chest in the time period of the FCV. The time domain waveforms of FCV and spontaneous cough have been published and are freely available for a multiplicity of biometric measures and disease conditions. A sketch of the key phases of FCV in the time domain may be illustrated in FIG. 1B.

Accordingly, in some embodiments, a sound signal data signature analysis platform 100 may employ the SDS analysis service 110 to analyze sound signal data signatures from one or more recording devices 104, e.g., via a network 10. In some embodiments, the network 10 may include any suitable computer network, including, two or more computers that are connected with one another for the purpose of communicating data electronically. In some embodiments, the network 10 may include a suitable network type, such as, e.g., a local-area network (LAN), a wide-area network (WAN) or other suitable type. In some embodiments, a LAN may connect computers and peripheral devices in a physical area, such as a business office, laboratory, or college campus, by means of links (wires, Ethernet cables, fiber optics, wireless such as Wi-Fi, etc.) that transmit data. In some embodiments, a LAN may include two or more personal computers, printers, and high-capacity disk-storage devices called file servers, which enable each computer on the network 10 to access a common set of files. In some embodiments, the network 10 may include, e.g., a network of networks, such as devices on a LAN networked with other LANs or one or more WANs, such as, e.g., the Internet. In some embodiments, a WAN may connect computers and smaller networks to larger networks over greater geographic areas. A WAN may link the computers by means of cables, optical fibers, or satellites, or other wide-area connection means. In some embodiments, an example of a WAN may include the Internet.

In some embodiments, the SDS analysis service 110 may be implemented using any suitable computing device or computing system or both. For example, the SDS analysis service 110 may include, e.g., a server or server system. In some embodiments, a server refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user). The aforementioned examples are, of course, illustrative and not restrictive.

In some embodiments, the recording device(s) 104 may include a signal data signature recording engine 120 to capture audio recordings of FCV and produce SDS for each FCV. The SDS and/or the FCV may then be uploaded, via the network 10, to the SDS analysis service 110 for recognition tasks among other analysis of the user's FCV.

In some embodiments, the SDS analysis service 110 may include a full ML monitoring system that includes a visual navigation system. The visual navigation system allows millions of audio files to be visualized and clustered in high-dimensional space. The incoming data-stream is transformed into a feature vector and projected into this high-dimensional space such that it is fully characterized using an unsupervised and/or semi-supervised learning based off of the millions of audio feature vector data points from a training dataset such that all data attributes are automatically characterized.

In some embodiments, once a high-quality audio file is identified it may be transformed into an image file or spectrogram using specialized processing tools and approaches. In some embodiments, a specialized set of filter banks that capture the frequency signatures and time concordance of the audio files are used to create a distinctive feature set.

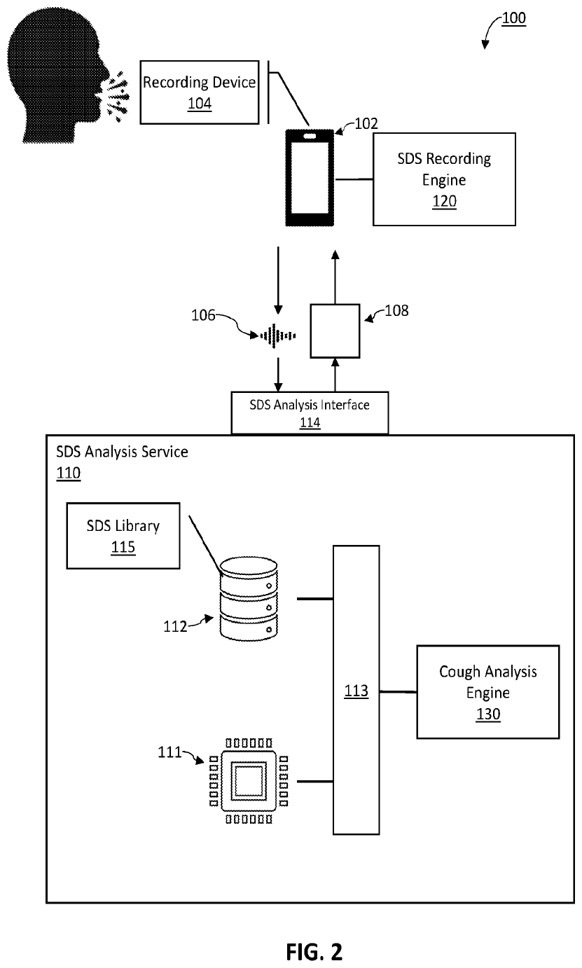

FIG. 2 depicts an illustrative computer-based system for sound signal data signature analysis including a sound signal data signature analysis service that receives sound signal data signatures from a user computing device according to embodiments of the present disclosure.

In some embodiments, a user may record a sound signal data signature using a recording device 104 of a user computing device 102. For example, the user computing device 102 may include one or more microphones and a software application configured to use the microphones for recording sounds. However, in some embodiments, the recording device 104 may be a peripheral or connected device connected to the user computing device 102, and the user computing device 102 may include a software application configured to receive or obtain a recording from the recording device 104.

In some embodiments, the sound signal data signature may include a forced non-speech vocalization, such as, e.g., a cough. As described above, a sound signature of a forced non-speech vocalization is unique to each individual and varies according to the respiratory condition and/or health of the individual. Thus, the user computing device 102 may instruct the user to force a cough vocalization, e.g., as a way to authenticate a user's identity. The sound signal data signature may also be used to assess changes to the sound signature of the user's sound signal data signature by, e.g., comparing the sound signal data signature to a baseline signature. Thus, the sound signal data signature may be employed to assess any potential changes to the user's sound signal data signature that may indicate a potential respiratory anomaly.

In some embodiments, the SDS analysis service 110 may be configured to receive the forced cough vocalization 106 and compare a signature thereof to a baseline signature in order to identify anomalous sound signal data signatures may provide more efficient early warning and screening for any anomaly inducing factor, improving the speed, efficiency, cost and access to quickly recognizing and mitigating the anomaly inducing factor.

Accordingly, in some embodiments, the user computing device 102 may capture the audio data of the FCV from the recording device and formulate an SDS using an SDS recording engine 120. In some embodiments, the SDS recording engine 120 may determine a sound signal data signature recording of the sound signal data signature isolated from noise and artifacts of in the recorded forced cough vocalization 106, generate a signature for the sound signal data signature recording,

In some embodiments, the user computing device 102 may then provide the forced cough vocalization 106 recorded by the recording device 104, including the SDS, to the SDS analysis service 110, e.g., via a sound signal data signature analysis interface 114. In some embodiments, the sound signal data signature analysis interface 114 may include any suitable interface for data communication over, e.g., a network (e.g., network 10 described above), or via local or direct data communication infrastructure. For example, in some embodiments, the sound signal data signature analysis interface 114 may include wired interfaces such as, e.g., a Universal Serial Bus (USB) interface, peripheral component interconnect express (PCIe), serial AT attachment (SATA), or any other wired interface, or wireless interfaces such as, e.g., Bluetooth™, near-field wireless communication (NFC), RFID, Narrow Band Internet of Things (NBIOT), 3G, 4G, 5G, GSM, GPRS, WiFi, WiMax, CDMA, satellite, ZigBee, or other wireless interface, or any combination of any wired and/or wireless interfaces. In some embodiments, the user computing device 102 may communicate the forced cough vocalization 106 via the sound signal data signature analysis interface 114 using any suitable data communication protocol, such as, e.g., IPX/SPX, X.25, AX.25, AppleTalk™, TCP/IP (e.g., HTTP), application programming interface (API), messaging protocol or any combination thereof.

In some embodiments, the sound signal data signature analysis interface 114 may include, e.g., an application programming interface. In some embodiments, “application programming interface” or “API” refers to a computing interface that defines interactions between multiple software intermediaries. An “application programming interface” or “API” defines the kinds of calls or requests that can be made, how to make the calls, the data formats that should be used, the conventions to follow, among other requirements and constraints. An “application programming interface” or “API” can be entirely custom, specific to a component, or designed based on an industry-standard to ensure interoperability to enable modular programming through information hiding, allowing users to use the interface independently of the implementation.

In some embodiments, the SDS analysis service 110 may receive the forced cough vocalization 106 and the associated SDS to analyze the forced cough vocalization 106 and analyze the SDS, e.g., relative to a baseline signature. In some embodiments, the SDS analysis service 110 may be a part of the user computing device 102. Thus, the SDS analysis service 110 may include hardware and software components including, e.g., user computing device 102 hardware and software, cloud or server hardware and software, or a combination thereof.

In some embodiments, the SDS analysis service 110 may include hardware components such as a processor 111, which may include local or remote processing components. In some embodiments, the processor 111 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor 111 may include data-processing capacity provided by the microprocessor. In some embodiments, the microprocessor may include memory, processing, interface resources, controllers, and counters. In some embodiments, the microprocessor may also include one or more programs stored in memory.

Similarly, the SDS analysis service 110 may include data store 112, such as local hard-drive, solid-state drive, flash drive, database or other local storage, or remote storage such as a server, mainframe, database or cloud provided storage solution. In some embodiments, the data storage solution of the data store 112 may include, e.g., a suitable memory or storage solutions for maintaining electronic data representing the activity histories for each account. For example, the data storage solution may include database technology such as, e.g., a centralized or distributed database, cloud storage platform, decentralized system, server or server system, among other storage systems. In some embodiments, the data storage solution may, additionally or alternatively, include one or more data storage devices such as, e.g., a hard drive, solid-state drive, flash drive, or other suitable storage device. In some embodiments, the data storage solution may, additionally or alternatively, include one or more temporary storage devices such as, e.g., a random-access memory, cache, buffer, or other suitable memory device, or any other data storage solution and combinations thereof.

In some embodiments, the SDS analysis service 110 may implement computer engines, including an SDS analysis engine 130 to leverage machine learning models generate a signature for the sound signal data signature recording, to leverage machine learning models compare the signature to a baseline signature to identify potentially anomalous sound signal data signatures, and/or to generate an authentication determination of the user based on deviations for the signature of the sound signal data signature recording from the baseline signature, among other analysis functions. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the SDS recording engine 120 is depicted as a local computer engine that is local to the user computing device 102, while the SDS analysis engine 130 is depicted as remote from the user computer device 102 in the SDS analysis service 110. However, other arrangements are contemplated, including both the SDS recording engine 120 and the SDS analysis engine 130 being local to the user computing device 102, remote from the user computing device 102, or provided in a hybrid implementation with some functions and/or features of the SDS recording engine 120 and/or the SDS analysis engine 130 being implemented locally, and others remotely.

In some embodiments, the first component is the mobile device application on the user computing device 102. In some embodiments, the mobile device application may exist as a native Android application, a native iOS application, a native Windows application, and/or a web client application or any other suitable application or any combination thereof. The mobile application records audio at a suitable bit rate and bit depth, such as, e.g., 32 bit float 48 k bit full spectrum sound, and sends that sound to, e.g., a server configured as the SDS analysis service 110, as an uncompressed file, as a compressed lossless file, as a compressed lossy file, or according to any suitable file type and file format or any combination thereof.

In some embodiments, the SDS recording engine 120 receive and record the sound file (e.g., the forced cough vocalization 106), and then place it through a series of audio filters to force mono compatibility, standardize signal level and remove ancillary noise. Additionally, any other suitable filters may be employed for signal quality optimization, such as one or more filters for, e.g., dynamic range modification (e.g., via dynamic range compression or expansion), optimization of signal to noise ratio, removal, suppression or other mitigation of ancillary noise(s), implement bandlimiting to isolate frequency content within a range of interest (e.g., via resampling or the use of equalization filters), among other signal optimizations or any combination thereof.

In some embodiments, the SDS recording engine 120 may leverage local recording devices 104 for audio capture of FCV in an SDS, transfer the SDS to network computer-based system and platform to store, classify, evaluate, filter, and then repeat FCV local recording devices for audio capture for ideal SDS capture without limitation of SDS frequency, audio segment duration, data storage size or data storage duration, or repeatability of FCV capture.

In some embodiments, the SDS recording engine 120 may inform the user, e.g., via graphical user interface (GUI) on a computing device 102, to conduct an FCV with visual and or audio queues on the local recording device or device screen. And SDS can contain the audio signature and recording of one or multiple FCVs. And an SDS segment will contain only one FCV audio signature and recording.

In some embodiments, the SDS recording engine 120 may request, e.g., via the GUI, the user to provide FCVs at a particular cadence or timing for multiple FCVs within a single SDS using visual and or audio queues. For example, the GUI may present a visual indicator that appears and/or presents a particular appearance when the user is requested to provide a next FCV in the multiple FCVs. Alternatively, or in addition, the GUI may present an instruction to the user to provide an FCV once every predetermined period, e.g., once every 1, 2, 3, 4, 5, 6, 7, 8, 9, 10, 15, 20, 25, 30 or more seconds.

In some embodiments, the SDS recording engine 120 may then submit the SDS and SDS segments to the SDS analysis service 110 for storage in the data store 112 and evaluation by the SDS analysis engine 130.

In some embodiments, the SDS recording engine 120 may request reinvocation of a FCV, which can be triggered and/or executed based on the visual and audio queues in relation to an optimal criteria of the SDS not being met due to, but not limited to, background noise, poor audio quality, recording or storage failure, network error or improper phasing of the cough, and the like, or some combination thereof. Within a given SDS, there can be many instances of parasitic information such as, but not limited to: background noise, speech, and overall non-cough sounds that can be ascertained by software systems and audio filtering mechanisms that analyze the audio signature, spectrum, or spectrogram affiliated with the SDS or SDS segments, and the like. In some embodiments, after filtering the sound signal data signature file may be passed to one or more machine learning models of the SDS analysis engine 130 to function as an AI sound detector. In some embodiments, this sound detector may be trained, utilizing calibration quality professionally audio engineered sound source libraries to differentiate a forced non-speech vocalization from other vocal and non-vocal sounds. The sound detector may provide a probability score that the that the incoming sound is a match to the target sound source library and not a match to the not target sound source library.

In some embodiments, the baseline match value cluster may be established at setup where the SDS analysis service 110 collects multiple baseline sound signal data signatures (e.g., two, three, five, six, less than six, more than six or other suitable number) according to the process described above. For example, the SDS analysis service 110 may collect sound signal data signatures from the user computing device 102, perform audio analysis to isolate and extract the sound signal data signature records using the audio filters of the SDS recording engine 120, generate sound signal data signatures for each sound signal data signature using the sound detector of the SDS analysis engine 130, and classify each sound signal data signature by computing match values with an SDS classifier. In some embodiments, the cluster formed by the match values of the sound signal data signatures at set-up may be tested against as baseline sound signatures.

In some embodiments, the SDS analysis engine 130 may be configured to utilize one or more exemplary AI/machine learning techniques as an AI-based sound detector. The AI/machine learning technique may be, e.g., chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, one or more hidden Markov models, Gaussian 22 mixture models, Bayesian models, clustering algorithms, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
a. define Neural Network architecture/model,
b. transfer the input data to the exemplary neural network model,
c. train the exemplary model incrementally,
d. determine the accuracy for a specific number of timesteps,
e. apply the exemplary trained model to process the newly-received input data,
f. optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, as a result of the machine learning-based processing of the SDS, the SDS analysis engine 130 may output a determination, such as, e.g., an anomaly determination 108. In some embodiments, the anomaly determination 108 may include, e.g., a label and/or probability value of a classification of the SDS as anomalous, e.g., relative to a set of baseline SDS of the user, e.g., stored in an SDS library 115 of the data store 112. In some embodiments, the sound detector may be trained to classify the SDS as anomalous or non-anomalous based on the set of baselines in the SDS library 115. For example, the set of baselines may be collected during an initial onboarding of the user to establish a healthy and/or authentic signature for forced cough vocalizations. Thus, by predicting an anomaly determination 108 for each baseline and computing an error based on a deviation of the anomaly determination 108 from a classification as healthy and/or authentic, the SDS analysis engine 130 may train parameters of the sound detector to more accurately identify a non-anomalous SDS, and thus more accurately identify an anomalous SDS.

In some embodiments, the anomaly determination 108 may be output, e.g., to the user computing device 102 via the SDS analysis interface 114. The SDS analysis interface 114 may format the anomaly determination 108 to cause the user computing device 102 to present, e.g., via the GUI, the anomaly classification to the user. For example, the user computing device 102 may use the GUI and the anomaly determination 108 to provide an alert or warning of an anomalous SDS, and thus a potential health and/or authentication concern.

In some embodiments, the SDS analysis service 110 may output the anomaly determination 108 to one or more other devices and/or systems, such as, e.g., a hardware and/or software access control to permit or deny access based on the SDS being anomalous or non-anomalous, or to a physician or pharmacist computing device to alert a patient care provider of a potentially unhealthy condition, and/or to one or more other devices and/or systems or any combination thereof.

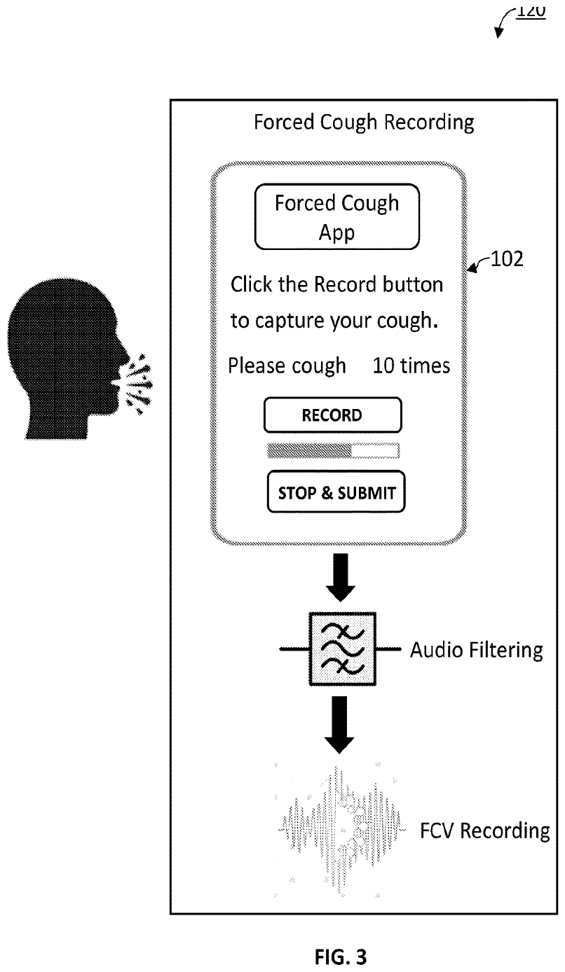

FIG. 3 depicts an illustrative computer-based system for FCV recording including a user computing device configured to record sound FCV recording signatures of a user according to embodiments of the present disclosure.

In some embodiments, the SDS recording engine 120 of the user computer device 102 may interact with the GUI of the user computing device 102 to instruct the user to provide one or more FCVs. In some embodiments, the SDS recording engine 120 may be configured to cause the GUI present one or more user interface (UI) elements to present the instruction to the user to produce the FCV(s). In some embodiments, the FCV(s) may include a series of multiple FCVs at predetermined intervals. Accordingly, the instruction presented by the GUI may include instructions for when and how to provide an FCV according to the predetermined interval, a visual and/or audible indicator to prompt the user for an FCV at each predetermined interval, or any other suitable instruction or any combination thereof.

In some embodiments, the SDS recording engine 120 may also interact with the recording device 104 to record the FCVs produced by the user. For example, the SDS recording engine 120 may control the recording device 104 to continuously record all of the one or more FCVs throughout each predetermined interval. In another example, the SDS recording engine 120 may control the recording device 104 to commence recording at a point before each predetermined interval, and end recording at a point after each predetermined interval to individually record the one or more FCVs.

Accordingly, in some embodiments, the user computing device 102 may record audio, e.g., in mono or stereo or both. The audio may be filtered to remove unwanted noise, such as, e.g., environmental noises, speech, animal sounds, among other noises that are not related to the forced cough. Thus, the SDS recording engine 120 may filter the recordings to produce an FCV recording including filtered audio of each FCV.

To do so, the SDS recording engine 120 may employ one or more audio filters. For example, human physiology (lung structure and nonsegmental tracheobronchial lengths combine to provide frequency harmonics in the range of 200 Hz to 48,000 Hz). In turn FCV and auscultation energy and information may be in the range of 200 Hz to 48,000 Hz. Thus, the audio filters may include filters configured to remove noise outside of the range of 200 Hz to 48,000 Hz.

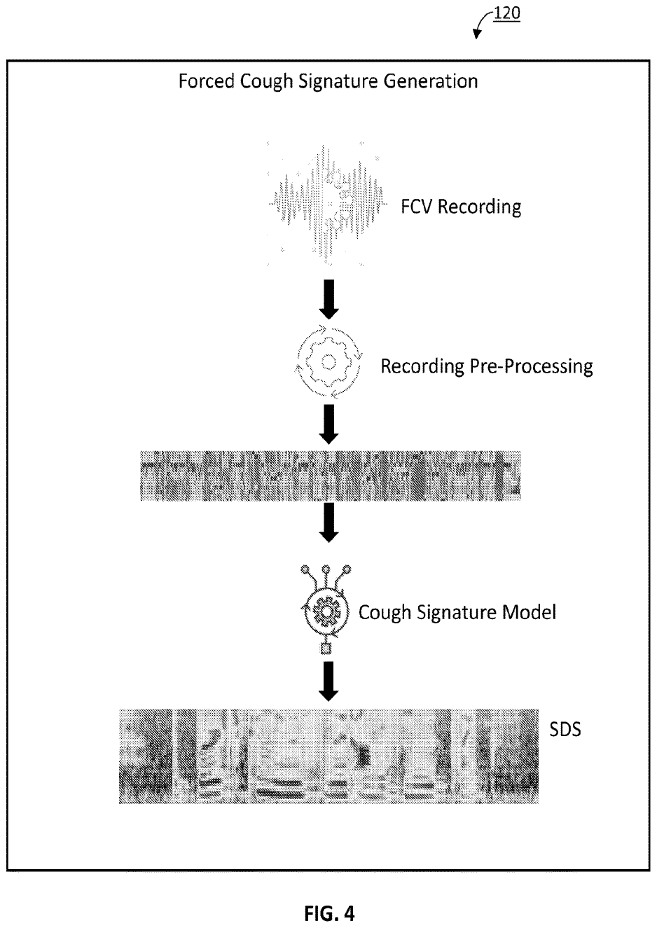

FIG. 4 depicts an illustrative computer-based system for SDS generation from the FCV recording according to embodiments of the present disclosure.

In some embodiments, upon generation of the FCV recording, the SDS recording engine 120 may process the audio of the FCV recording to extract signal data from the FCV recording. To do so, the SDS recording engine 120 may pre-process the audio of the FCV recording. In some embodiments, the signal data may be any suitable data representative of an audio signal of each FCV recording. Such signal data may include, e.g., amplitude data, frequency data, spectral data, dynamic range, sample rate, among other time-dependent and/or time-independent data representative of the FCV recording or any combination thereof.

In some embodiments, pre-processing may impose standards upon the FCV recording via one or more cleansing, filtering and/or normalizing processes. Such cleansing, filtering and/or normalizing ensures high-quality audio files. These filters act to address concerns regarding audio quality for processing, such as, e.g., stereo to mono compatibility, peak input loudness level, and attenuation of unrelated low frequencies or other ancillary noise. Additionally, any other suitable filters may be employed for signal quality optimization, such as one or more filters for, e.g., dynamic range modification (e.g., via dynamic range compression or expansion), optimization of signal to noise ratio, removal, suppression or otherwise mitigation of ancillary noise(s), implementation of bandlimiting to isolate frequency content within a range of interest (e.g., via resampling or the use of equalization filters), among other signal optimizations or any combination thereof . . . For example, background noise may be filtered from a sample including one or more recordings of a vocalization, and then the vocalization with the recording(s) can be identified, e.g., using a Pretrained Audio Neural Network (PANN) or other detection/recognition tools or any combination thereof. Thus, audio samples that do not contain a vocalization may be prevented from being processed by the system to avoid unnecessary resource utilization.

For example, in some embodiments, the recording pre-processing may include, e.g., bandpass filtering, up-sampling, down-sampling, conversion between mono and stereo, generation of a spectrograph, generation of a waveform representation of the FCV recording, among other pre-processing functions.

In some embodiments, the signal data may be analyzed by a cough signature model. In some embodiments, the cough signature model may include one or more algorithms configured to extract the SDS from the signal data. In some embodiments, the cough signature model may include, e.g., cropping, trimming, or otherwise reshaping a representation of the FCV recording (e.g., a waveform, spectrogram, etc.), or any combination thereof. The reshaping may remove non-FCV-related data in order to extract the data associated with the FCV itself. In some embodiments, such non-FCV-related data may include, e.g., noise, a portion without the forced cough, other non-cough sounds, among other non-FCV-related data or any combination thereof. Thus, the cough signature model identifies the FCV and isolates the data associated with the FCV.

In some embodiments, the FCV recording may include data representative of the multiple FCVs. For example, the recording device 104 may continuously record while the user is instructed to produce multiple FCVs at predetermined intervals. Accordingly, the SDS recording engine 120 may use the cough signature model to isolate and extract the data of each cough in the FCV recording, thus extracting segments of the SDS, where each segment is an individual cough in the FCV recording.

In some embodiments, the cough signature model may include, e.g., one or more AI/machine learning techniques, such as those detailed above. Accordingly, the cough signature model may include parameters that have been trained based on historical training data where portions of one or more signals are known to be FCV-related or non-FCV-related (e.g., annotated FCV signal data). The cough signature model may produce a prediction of an extracted SDS from the annotated FCV signal data, and then, based on a error between the annotations of the annotated FCV signal data and the prediction of extracted SDS, the parameters of the cough signature model may be updated to better correlate signal data to FCV-related data.

Alternatively, or in addition, the cough signature model may include one or more processing algorithms, including, e.g., thresholding, Fourier transform or other transform, among other algorithms or any combination thereof. As a result, the SDS recording engine 120 may produce an SDS for the FCV recording from the user.

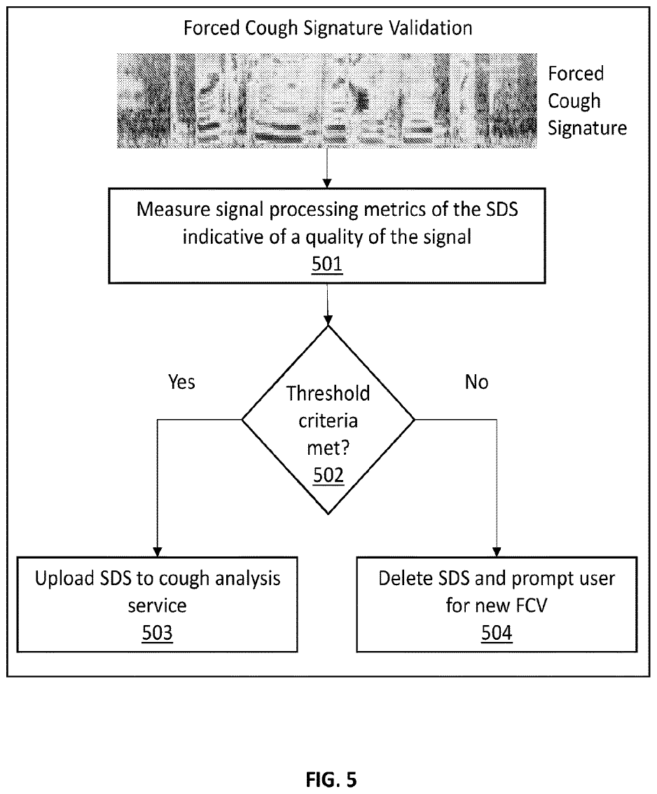

FIG. 5 depicts an illustrative system for the SDS recording engine 120 including one or more components configured for SDS validation to validate the SDS in accordance with one or more embodiments of the present disclosure.

Human physiology (lung structure and nonsegmental tracheobronchial lengths combine to provide frequency harmonics in the range of 200 Hz to 48,000 Hz). In turn FCV and auscultation energy and information may be in the range of 200 Hz to 48,000 Hz.

In some embodiments, voluntary forced cough (forced cough vocalization or FCV) and auscultation has the advantage of being delivered on demand and reproducible and available to recording systems such as microphone, computer with microphone, phone or mobile phone. Auscultation is typically completed by a medical professional as part of the evaluation of FCV and fail to capture the breath of the frequency range, features in turn a comparison of FCV based SDSs in the complete frequency range.

In some embodiments, a Signal Data Signature may include a sample recording of a continuous acoustic signal from FCVs. Signal Data Signature classification has different commercial applications such as unobtrusive monitoring and diagnosing in health care and medical diagnostics. An SDS may or may not include unique sound features that are pathognomonic for chronic exposures, unchanged by differences in burst energy from the source signal data after the process of capturing, extracting and feature engineering.

In some embodiments, some recording systems may be able to capture SDS across a complete audio signal across a complete frequency range. However, such systems lack the ability to detect the FCV in the SDS or SDS segment, have limited duration, and or limited repeatability.

In some embodiments, auscultation of FCV using recording systems has the advantage of a signal data signature detection with maximum signal frequency range and signal fidelity and SDS acquisition repeatability for the purpose pf providing training, testing, and evaluation of FCV to classify and identify features (such as disease).

In some embodiments, the present disclosure provides technical solutions to technical problems including the require human input and human decision points, algorithms that fail to capture the source signal data signature and unable to perform well on datasets that were not present during Artificial Intelligence and Machine Learning training, testing, and evaluation. 29

In some embodiments, the SDS recording engine 120 may leverage local recording devices 104 for audio capture of FCV in an SDS, transfer the SDS to network computer-based system and platform to store, classify, evaluate, filter, and then repeat FCV local recording devices for audio capture for ideal SDS capture without limitation of SDS frequency, audio segment duration, data storage size or data storage duration, or repeatability of FCV capture.

In some embodiments, the SDS recording engine 120 may inform the user, e.g., via graphical user interface (GUI) on a computing device 102, to conduct an FCV with visual and or audio queues on the local recording device or device screen. And SDS can contain the audio signature and recording of one or multiple FCVs. And an SDS segment will contain only one FCV audio signature and recording.

In some embodiments, the SDS recording engine 120 may request, e.g., via the GUI, the user to provide cadence or timing for multiple FCVs within a single SDS using visual and or audio queues.

In some embodiments, the SDS recording engine 120 may then submit the SDS and SDS segments to a network computer-based system for storage and evaluation.

In some embodiments, the SDS recording engine 120 may request reinvocation of a FCV through the visual and audio queues is optimal criteria of the SDS is not met due to background noise, poor audio quality, recording or storage failure, network error or improper phasing of the cough (note: phase filter embodiment).

Accordingly, in some embodiments, the SDS recording engine 120 may test the SDS against one or more criteria indicative of the quality of the recording of a forced cough vocalization. To do so, the SDS recording engine 120 may implement, at block 501, one or more signal processing metrics to measure the quality of the SDS. In some embodiments, the signal processing metrics may include, e.g., signal-to-noise ratio, noise floor, clipping, sibilance, speech, or specific non-cough sounds (e.g., a car horn beeping, a door closing, a baby crying, for example), noise with pitch outside of the human creatable pitch range (e.g., 85 Hz to 155 Hz), among other signal quality measurements or any combination thereof. In some embodiments, the SDS recording engine 120 may utilize thresholds for each signal processor metric, where if the SDS fails to achieve the threshold, the SDS is discarded and the process is restarted, but where the SDS does achieve the threshold, the SDS is uploaded to the cough analysis service 110. In some embodiments, the threshold may include, e.g., a maximum signal-to-noise ratio, a maximum noise floor, a maximum quantity of clipping, a maximum sibilance, among other signal quality measurements or any combination thereof.

In some embodiments, the SDS recording engine 120 may implement criteria associated with system and/or device status, error, or other functionality or any combination thereof. For example, the SDS recording engine 120 may monitor error logs and system logs to determine operating status of, e.g., the recording device 104, one or more processing resources of the user computing device 102 (e.g., a processor, a memory, a storage device, a wireless radio, a hardware interface, among other component or any combination thereof), one or more software resources of the user computing device 102 (e.g., program faults and/or errors, among others or any combination thereof. In some embodiments, where the SDS recording engine 120 determines that the system and/or device status, error, or other functionality or any combination thereof is indicative of an inability of the user computing device 102 to capture a complete FCV, the criteria may be automatically determined to not be met.

In some embodiments, the SDS recording engine 120 may implement criteria associated with phases of an FCV (see, e.g., FIG. 1A detailed above). In some embodiments, as detailed above with respect to FIG. 1A, a forced cough may have three phases, an explosive phase, an intermediate phase, and a voiced phase. The phases may be identified based on time-varying frequency of the SDS. For example, each phase may have an associated range of frequencies, where if the SDS includes data indicative of a frequency outside of the range of a particular phase, the particular phase may be deemed missing or obfuscated, and thus the SDS may fail to meet the phase criteria.

For example, in some embodiments, in order to ensure maximum data quality (e.g., determine that the cough is a high quality cough), a filter is implemented in order to measure whether the SDS contains all three phases of a cough. According to some embodiments, since all cough sounds contain the first two phases, the filter may operate to determine, gauge, ensure or otherwise identify whether the third phase is present (e.g., the voiced phase of the cough). In some embodiments, detection of the third phase can involve a determination as to whether the third phase includes a threshold satisfying pitch.

Therefore, according to some embodiments, as discussed in more detail below, the disclosed systems and methods can implement a pitch detection algorithm (PDA). The PDA, which can operate in the time domain and/or frequency domain, can be utilized to determine or identify the pitch or fundamental frequency of a quasiperiodic and/or oscillating signal from the cough (e.g., a digital recording of the cough and/or direct “speech” input of the cough). In some embodiments, the PDA can be any type of known or to be known PDA, such as, but not limited to, frequency-domain PDA algorithms, spectral/temporal PDA algorithms, speech detection algorithms, and the like, or some combination thereof.

According to some embodiments, the upon the PDA analysis, the filter can determine whether the to send the cough for classification (e.g., upload the SDS to the cough analysis service 110). For example, in some embodiments, if a SDS segment and associated cough sound has a certain threshold of pitch in its third phase, it is sent downstream for classification. Conversely, if a cough contains minimal to no pitch (e.g., a pitch below the pitch threshold), it is filtered out, and not sent downstream for further preprocessing and ultimately the neural networks.

In some embodiments, the SDS recording engine 120 may, at block 502, evaluate the criteria, including determining whether threshold criteria, e.g., for the signal processing metrics detailed above, are met by the SDS.

In some embodiments, where the criteria are met, the SDS may be communicated, at block 503, to a downstream analysis, such as, e.g., a machine learning model, post-processing step, storage, or other analysis component in a local and/or remote implementation. For example, the SDS recording engine 120 may upload the SDS to the cough analysis service 110.

In some embodiments, where the criteria are not met, the SDS may be deleted, e.g., from a memory of the user computing device 102, and the SDS engine 120 may control the GUI and/or the recording device to instruct the user to provide one or more new FCVs and to record the one or more new FCVs, e.g., via a process as detailed above.

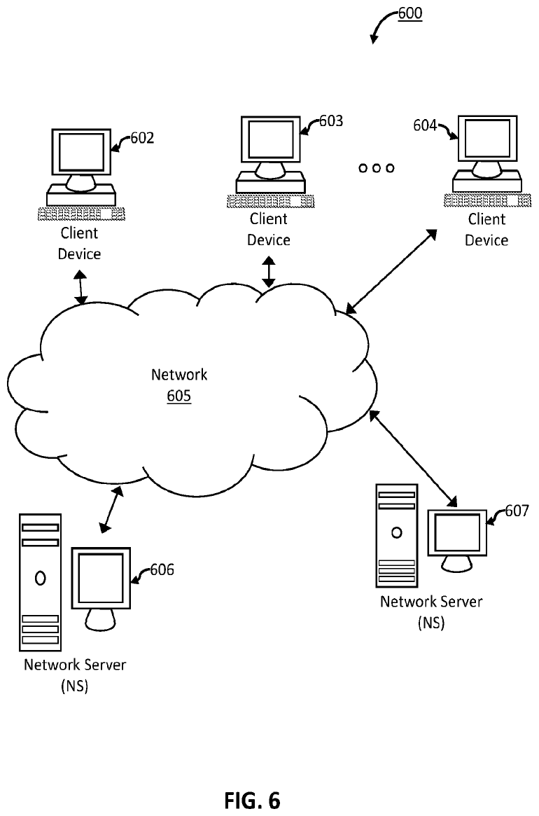

FIG. 6 depicts a block diagram of an exemplary computer-based system and platform 600 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 600 may be configured to manage a large number of members and concurrent transactions, as detailed herein. In some embodiments, the exemplary computer-based system and platform 600 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 6, member computing device 602, member computing device 603 through member computing device 604 (e.g., clients) of the exemplary computer-based system and platform 600 may include virtually any computing device capable of receiving and sending a message over a network (e.g., cloud network), such as network 605, to and from another computing device, such as servers 606 and 607, each other, and the like. In some embodiments, the member devices 602-604 may be personal computers, multiprocessor systems, microprocessor-based or programmable consumer electronics, network PCs, and the like. In some embodiments, one or more member devices within member devices 602-604 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 602-604 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 602-604 may include may run one or more applications, such as Internet browsers, mobile applications, voice calls, video games, videoconferencing, and email, among others. In some embodiments, one or more member devices within member devices 602-604 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 602-604 may be specifically programmed by either Java, . Net, QT, C, C++ and/or other suitable programming language. In some embodiments, one or more member devices within member devices 602-604 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 605 may provide network access, data transport and/or other services to any computing device coupled to it. In some embodiments, the exemplary network 605 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 605 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 605 may include and implement, as an alternative or in conjunction with one or more of the above, a WiMAX architecture defined by the WiMAX forum. In some embodiments and, optionally, in combination of any embodiment described above or below, the exemplary network 605 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 605 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 605 may also include mass storage, such as network attached storage (NAS), a storage area network (SAN), a content delivery network (CDN) or other forms of computer or machine readable media.

In some embodiments, the exemplary server 606 or the exemplary server 607 may be a web server (or a series of servers) running a network operating system, examples of which may include but are not limited to Microsoft Windows Server, Novell NetWare, or Linux. In some embodiments, the exemplary server 606 or the exemplary server 607 may be used for and/or provide cloud and/or network computing. Although not shown in FIG. 6, in some embodiments, the exemplary server 606 or the exemplary server 607 may have connections to external systems like email, SMS messaging, text messaging, ad content providers, etc. Any of the features of the exemplary server 606 may be also implemented in the exemplary server 607 and vice versa.

In some embodiments, one or more of the exemplary servers 606 and 607 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 602-604.

In some embodiments and, optionally, in combination of any embodiment described above or below, for example, one or more exemplary computing member devices 602-604, the exemplary server 606, and/or the exemplary server 607 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

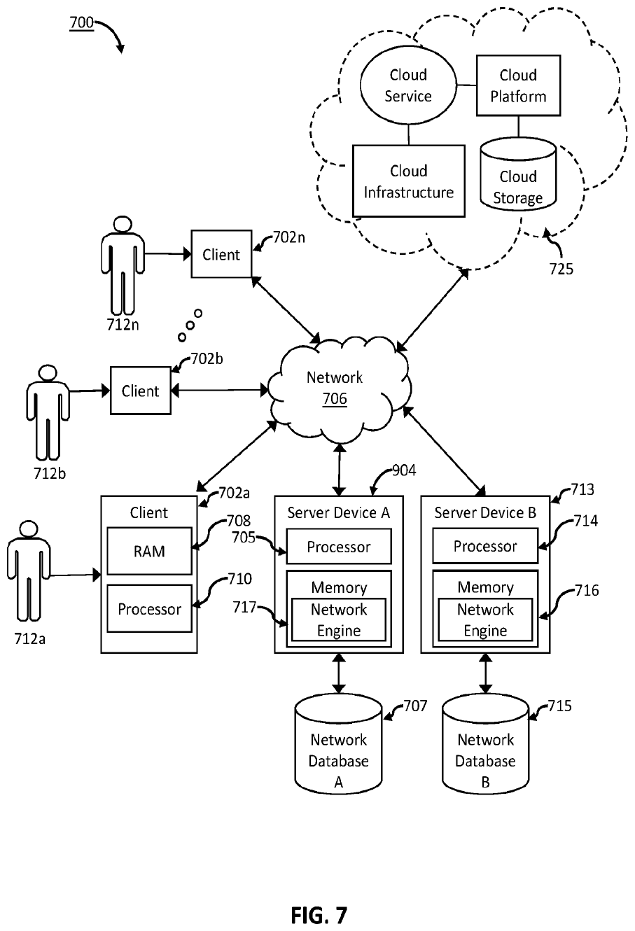

FIG. 7 depicts a block diagram of another exemplary computer-based system and platform 700 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing device 702a, member computing device 702b through member computing device 702n shown each at least includes a computer-readable medium, such as a random-access memory (RAM) 708 coupled to a processor 710 or FLASH memory. In some embodiments, the processor 710 may execute computer-executable program instructions stored in memory 708. In some embodiments, the processor 710 may include a microprocessor, an ASIC, and/or a state machine. In some embodiments, the processor 710 may include, or may be in communication with, media, for example computer-readable media, which stores instructions that, when executed by the processor 710, may cause the processor 710 to perform one or more steps described herein. In some embodiments, examples of computer-readable media may include, but are not limited to, an electronic, optical, magnetic, or other storage or transmission device capable of providing a processor, such as the processor 710 of client 702a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, member computing devices 702a through 702n may also comprise a number of external or internal devices such as a mouse, a CD-ROM, DVD, a physical or virtual keyboard, a display, or other input or output devices. In some embodiments, examples of member computing devices 702a through 702n (e.g., clients) may be any type of processor-based platforms that are connected to a network 706 such as, without limitation, personal computers, digital assistants, personal digital assistants, smart phones, pagers, digital tablets, laptop computers, Internet appliances, and other processor-based devices. In some embodiments, member computing devices 702a through 702n may be specifically programmed with one or more application programs in accordance with one or more principles/methodologies detailed herein. In some embodiments, member computing devices 702a through 702n may operate on any operating system capable of supporting a browser or browser-enabled application, such as Microsoft™, Windows™, and/or Linux. In some embodiments, member computing devices 702a through 702n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 702a through 702n, user 712a, user 712b through user 712n, may communicate over the exemplary network 706 with each other and/or with other systems and/or devices coupled to the network 706. As shown in FIG. 7, exemplary server devices 704 and 713 may include processor 705 and processor 714, respectively, as well as memory 717 and memory 716, respectively. In some embodiments, the server devices 704 and 713 may be also coupled to the network 706. In some embodiments, one or more member computing devices 702a through 702n may be mobile clients.

In some embodiments, at least one database of exemplary databases 707 and 715 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

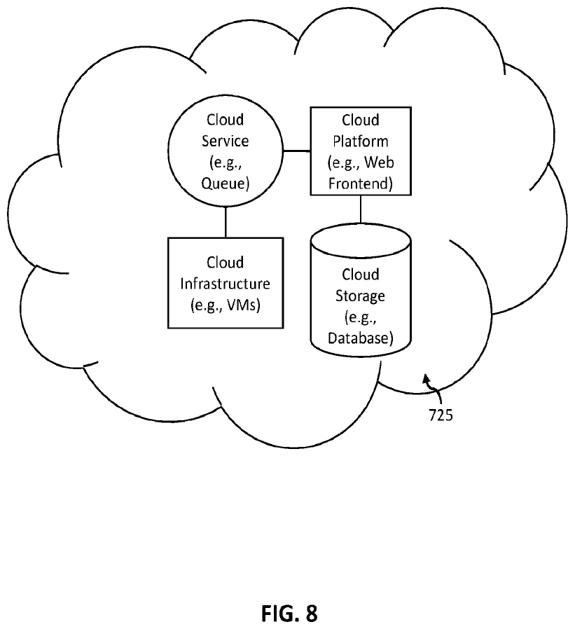

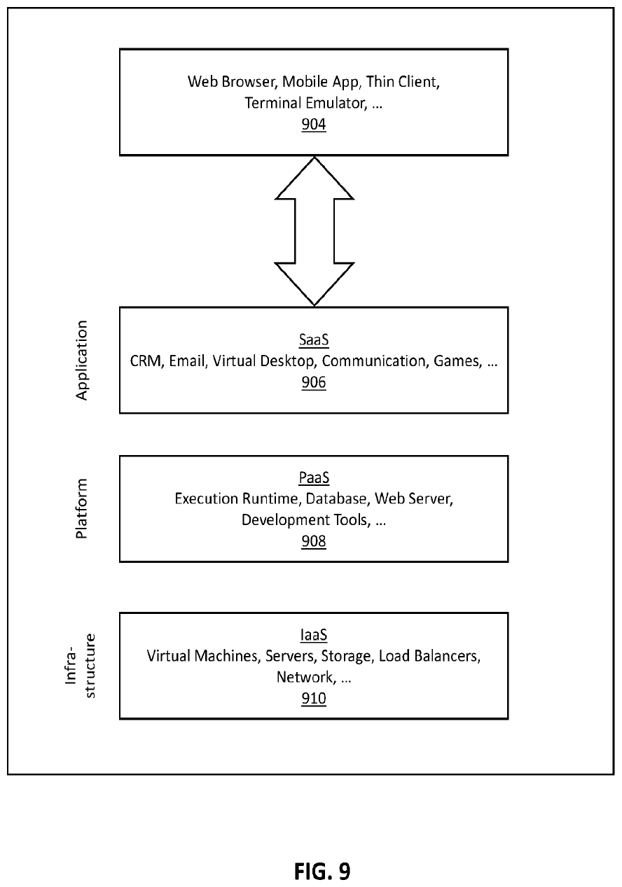

In some embodiments, the exemplary inventive computer-based systems/platforms, the exemplary inventive computer-based devices, and/or the exemplary inventive computer-based components of the present disclosure may be specifically configured to operate in a cloud computing/architecture 725 such as, but not limiting to: infrastructure a service (IaaS) 910, platform as a service (PaaS) 908, and/or software as a service (Saas) 906 using a web browser, mobile app, thin client, terminal emulator or other endpoint 904. FIGS. 8 and 9 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk(TM), TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, Javascript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999) , at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry ™, Pager, Smartphone, or any other reasonable mobile electronic device.

As used herein, terms “proximity detection,” “locating,” “location data,” “location information,” and “location tracking” refer to any form of location tracking technology or locating method that can be used to provide a location of, for example, a particular computing device, system or platform of the present disclosure and any associated computing devices, based at least in part on one or more of the following techniques and devices, without limitation: accelerometer(s), gyroscope(s), Global Positioning Systems (GPS); GPS accessed using Bluetooth™; GPS accessed using any reasonable form of wireless and non-wireless communication; WiFi™ server location data; Bluetooth ™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGS).

As used herein, the term “user” shall have a meaning of at least one user. In some embodiments, the terms “user”, “subscriber” “consumer” or “customer” should be understood to refer to a user of an application or applications as described herein, and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

The aforementioned examples are, of course, illustrative and not restrictive.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other Further still, the various steps may be carried out in any desired order (and any desired steps may be added, and/or any desired steps may be eliminated).

What is claimed is:

1. A method comprising:
controlling, by at least one processor, a display of a computing device to render a forced cough vocalization graphical user interface (GUI) comprising at least one user instruction instructing the user to auscultate and produce a forced cough vocalization;
controlling, by the at least one processor, a recording device associated with the computing device to record audio;
receiving, by the at least one processor from the recording device, at least one audio signal that captures the forced cough vocalization produced by the user;
utilizing, by the at least one processor, at least one pre-processing algorithm to pre-process the at least one audio signal and output forced cough vocalization signal data representative of the forced cough vocalization;
utilizing, by the at least one processor, at least one cough signature model to ingest the forced cough vocalization signal data and output a signal data signature representative of isolated forced cough vocalization-related signal data, wherein the isolated forced cough vocalization-related signal data is isolated from non-forced cough vocalization-related signal data;
wherein the at least one cough signature model to ingests the forced cough vocalization signal data and outputs the signal data signature based at least in part on at least one machine learning layer comprising a plurality of machine learning parameters;
wherein the plurality of machine learning parameters is iteratively refined based on error between:
previous predictions based on historical signal data signatures and known signal data signatures paired with the historical signal data signatures;
determining, by the at least one processor, a signal data signature invalidation based at least in part on testing of the signal data signature against at least one signal data signature criteria resulting in the signal data signature failing to achieve the at least one signal data signature criteria;
deleting, by the at least one processor, based on the signal data signature invalidation, the signal data signature; and
controlling, by the at least one processor, the display of the computing device to render the forced cough vocalization GUI comprising at least one subsequent user instruction instructing the user to auscultate and produce a new forced cough vocalization in response to the signal data signature invalidation.

2. The method of claim 1, further comprising:
controlling, by the at least one processor, the recording device associated with the computing device to record new audio;
receiving, by the at least one processor from the recording device, at least one new audio signal that captures the new forced cough vocalization produced by the user;
utilizing, by the at least one processor, the at least one pre-processing algorithm to pre-process the at least one new audio signal and output new forced cough vocalization signal data representative of the new forced cough vocalization;
utilizing, by the at least one processor, the at least one cough signature model to ingest the new forced cough vocalization signal data and output a at least one new signal data signature representative of new isolated forced cough vocalization-related signal data, wherein the new isolated forced cough vocalization-related signal data is isolated from new non-forced cough vocalization-related signal data;
determining, by the at least one processor, a signal data signature validation based at least in part on testing of the at least one new signal data signature against the at least one signal data signature criteria resulting in the signal data signature achieving the at least one signal data signature criteria; and
uploading, by the at least one processor, the at least one new signal data signature to a remote cough analysis service configured to utilize at least one machine learning model to detect at least one anomaly in the at least one new signal data signature.

3. The method of claim 2, wherein the remote cough analysis service further configured to store the at least one new signal data signature in a signal data signature library associated with the user so as to produce a set of baseline signal data signatures.

4. The method of claim 1, wherein the at least one signal data signature criteria comprises at least one of:
a maximum signal-to-noise ratio,
a maximum noise floor,
a minimum audio quality associated with the signal data signature,
a hardware failure,
a network error,
a cough sound or forced cough sound, and
a phasing criteria.

5. The method of claim 4, wherein the hardware failure comprises at least one of:
a failure in operation of the recording device, and
a failure in operation of a storage device associated with storing the at least one audio signal.

6. The method of claim 1, wherein the at least one processor is local to the computing device.

7. The method of claim 1, further comprising:
utilizing, by the at least one processor, the at least one cough signature model to ingest the forced cough vocalization signal data and output a plurality of signal data signatures representative of isolated forced cough vocalization-related signal data, wherein the isolated forced cough vocalization-related signal data is isolated for each of a plurality of forced cough vocalizations in the at least one audio signal.

8. A system comprising:
at least one processor configured to execute software instructions that, upon execution, cause the at least one processor to perform steps to:
control a display of a computing device to render a forced cough vocalization graphical user interface (GUI) comprising at least one user instruction instructing the user to produce a forced cough vocalization;
control a recording device associated with the computing device to record audio;
receive, from the recording device, at least one audio signal that captures the forced cough vocalization produced by the user for auscultation;
utilize at least one pre-processing algorithm to pre-process the at least one audio signal and output forced cough vocalization signal data representative of the forced cough vocalization;
utilize at least one cough signature model to ingest the forced cough vocalization signal data and output a signal data signature representative of isolated forced cough vocalization-related signal data, wherein the isolated forced cough vocalization-related signal data is isolated from non-forced cough vocalization-related signal data;
wherein the at least one cough signature model to ingests the forced cough vocalization signal data and outputs the signal data signature based at least in part on at least one machine learning layer comprising a plurality of machine learning parameters;
wherein the plurality of machine learning parameters is iteratively refined based on error between:
previous predictions based on historical signal data signatures and
known signal data signatures paired with the historical signal data signatures;
determine a signal data signature invalidation based at least in part on testing of the signal data signature against at least one signal data signature criteria resulting in the signal data signature failing to achieve the at least one signal data signature criteria;
delete based on the signal data signature invalidation, the signal data signature; and
control the display of the computing device to render the forced cough vocalization GUI comprising at least one subsequent user instruction instructing the user to auscultate and produce a new forced cough vocalization in response to the signal data signature invalidation.

9. The system of claim 8, wherein, upon execution of the software instructions, the at least one processor is further configured to:
control the recording device associated with the computing device to record new audio;
receive, from the recording device, at least one new audio signal that captures the new forced cough vocalization produced by the user;
utilize the at least one pre-processing algorithm to pre-process the at least one new audio signal and output new forced cough vocalization signal data representative of the new forced cough vocalization;
utilize the at least one cough signature model to ingest the new forced cough vocalization signal data and output a at least one new signal data signature representative of new isolated forced cough vocalization-related signal data, wherein the new isolated forced cough vocalization-related signal data is isolated from new non-forced cough vocalization-related signal data;
determine a signal data signature validation based at least in part on testing of the at least one new signal data signature against the at least one signal data signature criteria resulting in the signal data signature achieving the at least one signal data signature criteria; and
upload the at least one new signal data signature to a remote cough analysis service configured to utilize at least one machine learning model to detect at least one anomaly in the at least one new signal data signature.

10. The system of claim 9, wherein the remote cough analysis service further configured to store the at least one new signal data signature in a signal data signature library associated with the user so as to produce a set of baseline signal data signatures.

11. The system of claim 8, wherein the at least one signal data signature criteria comprises at least one of:
a maximum signal-to-noise ratio,
a maximum noise floor,
a minimum audio quality associated with the signal data signature,
a hardware failure,
a network error,
a cough sound or forced cough sound, and
a phasing criteria.

12. The system of claim 11, wherein the hardware failure comprises at least one of:
a failure in operation of the recording device, and
a failure in operation of a storage device associated with storing the at least one audio signal.

13. The system of claim 8, wherein the at least one processor is local to the computing device.

14. The system of claim 8, wherein, upon execution of the software instructions, the at least one processor is further configured to:
utilize the at least one cough signature model to ingest the forced cough vocalization signal data and output a plurality of signal data signatures representative of isolated forced cough vocalization-related signal data, wherein the isolated forced cough vocalization-related signal data is isolated for each of a plurality of forced cough vocalizations in the at least one audio signal.

15. A non-transitory computer readable medium having software instructions stored thereon, the software instructions configured to cause at least one processor to perform steps comprising:
control a display of a computing device to render a forced cough vocalization graphical user interface (GUI) comprising at least one user instruction instructing the user to auscultate and produce a forced cough vocalization;
control a recording device associated with the computing device to record audio;
receive, from the recording device, at least one audio signal that captures the forced cough vocalization produced by the user;
utilize at least one pre-processing algorithm to pre-process the at least one audio signal and output forced cough vocalization signal data representative of the forced cough vocalization;
utilize at least one cough signature model to ingest the forced cough vocalization signal data and output a signal data signature representative of isolated forced cough vocalization-related signal data, wherein the isolated forced cough vocalization-related signal data is isolated from non-forced cough vocalization-related signal data;
wherein the at least one cough signature model to ingests the forced cough vocalization signal data and outputs the signal data signature based at least in part on at least one machine learning layer comprising a plurality of machine learning parameters;
wherein the plurality of machine learning parameters is iteratively refined based on error between:
previous predictions based on historical signal data signatures and 56 known signal data signatures paired with the historical signal data signatures;
determine a signal data signature invalidation based at least in part on testing of the signal data signature against at least one signal data signature criteria resulting in the signal data signature failing to achieve the at least one signal data signature criteria;
delete based on the signal data signature invalidation, the signal data signature; and
control the display of the computing device to render the forced cough vocalization GUI comprising at least one subsequent user instruction instructing the user to auscultate and produce a new forced cough vocalization in response to the signal data signature invalidation.

16. The non-transitory computer readable medium of claim 15, wherein the software instructions are further configured to cause the at least one processor to perform steps comprising:
control the recording device associated with the computing device to record new audio;
receive, from the recording device, at least one new audio signal that captures the new forced cough vocalization produced by the user;
utilize the at least one pre-processing algorithm to pre-process the at least one new audio signal and output new forced cough vocalization signal data representative of the new forced cough vocalization;
utilize the at least one cough signature model to ingest the new forced cough vocalization signal data and output a at least one new signal data signature representative of new isolated forced cough vocalization-related signal data, wherein the new isolated forced cough vocalization-related signal data is isolated from new non-forced cough vocalization-related signal data; determine a signal data signature validation based at least in part on testing of the at least one new signal data signature against the at least one signal data signature criteria resulting in the signal data signature achieving the at least one signal data signature criteria; and
upload the at least one new signal data signature to a remote cough analysis service configured to utilize at least one machine learning model to detect at least one anomaly in the at least one new signal data signature.

17. The non-transitory computer readable medium of claim 16, wherein the remote cough analysis service further configured to store the at least one new signal data signature in a signal data signature library associated with the user so as to produce a set of baseline signal data signatures.

18. The non-transitory computer readable medium of claim 15, wherein the at least one signal data signature criteria comprises at least one of:
a maximum signal-to-noise ratio,
a maximum noise floor,
a minimum audio quality associated with the signal data signature,
a hardware failure,
a network error,
a cough sound or forced cough sound, and
a phasing criteria.

19. The non-transitory computer readable medium of claim 18, wherein the hardware failure comprises at least one of:
a failure in operation of the recording device, and
a failure in operation of a storage device associated with storing the at least one audio signal.

20. The non-transitory computer readable medium of claim 15, wherein the software instructions are further configured to cause the at least one processor to perform steps comprising:
utilize the at least one cough signature model to ingest the forced cough vocalization signal data and output a plurality of signal data signatures representative of isolated forced cough vocalization-related signal data, wherein the isolated forced cough vocalization-related signal data is isolated for each of a plurality of forced cough vocalizations in the at least one audio signal.
